(19) United States
(12) Patent Application Publication
(10) Pub. No.: US 2014/0235863 A1
(43) Pub. Date: Aug. 21, 2014

(54) SUBSTITUTED 4-ARYLTHIAZOLES AND PROCESS OF PREPARATION THEREOF

(71) Applicants: Supriya Singh, Lucknow (IN); Kuldeep Kumar Roy, Lucknow (IN); Sandeep Kumar Sharma, Lucknow (IN); Ranjana Srivastava, Lucknow (IN); Vinita Chaturvedi, Lucknow (IN); Anil Kumar Saxena, Lucknow (IN)

(72) Inventors: Supriya Singh, Lucknow (IN); Kuldeep Kumar Roy, Lucknow (IN); Sandeep Kumar Sharma, Lucknow (IN); Ranjana Srivastava, Lucknow (IN); Vinita Chaturvedi, Lucknow (IN); Anil Kumar Saxena, Lucknow (IN)

(73) Assignee: COUNCIL OF SCIENTIFIC & INDUSTRIAL RESEARCH, New Delhi (IN)

(21) Appl. No.: 14/117,415

(22) PCT Filed: Mar. 1, 2012
(86) PCT No.: PCT/IN12/00145

§ 371 Date: Nov. 13, 2013

(30) Foreign Application Priority Data
Jun. 3, 2011 (IN) 1580/DEL/2011

(51) Int. Cl. C07D 417/14; C07D 417/12; C07D 277/42

(52) U.S. Cl. CPC C07D 417/14; C07D 277/42; C07D 417/12
USPC 546/194; 548/193; 546/209

(57) ABSTRACT

The present invention relates to novel substituted 4-arylthiazoles, their preparation, and to their use as therapeutic agents, particularly in the prevention or treatment of tuberculosis. The present invention particularly relates to compounds of formula A:

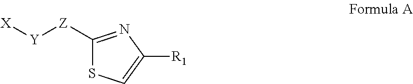

### FIELD OF INVENTION

The present invention relates to novel substituted 4-arylthiazoles, their preparation, and to their use as therapeutic agents, particularly in the prevention or treatment of tuberculosis. The present invention particularly relates to compounds of formula A:

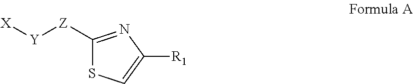

Wherein;
R₁ is substituted/unsubstituted aryl or heteroaryl group of the structure

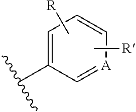

wherein, A is CH or N;

R and R′ are groups, which may be identical or different, selected from the group consisting of hydrogen, halogen, nitro, and methoxy,
X is a group selected from the group consisting of

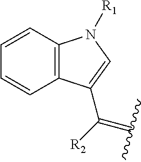

wherein, R₁ is a group selected from the group of benzyl and 2,4-dichlorobenzyl, while R2 is a group selected from hydrogen and methyl

or, a group of structure

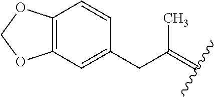 [SMILES: *=C(C)Cc1ccc2c(c1)OCO2]

or, a group of structure

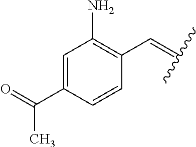 [SMILES: *=Cc1ccc(C(C)=O)cc1N]

or, a group of structure

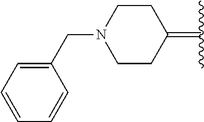 [SMILES: *=C1CCN(Cc2ccccc2)CC1]

or a group of structure

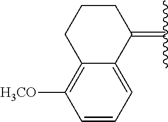 [SMILES: *=C1CCCc2c(OC)cccc21]

or a group of structure

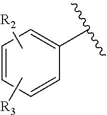

wherein, R2 and R3 may be same or different selected from the group of hydrogen, halogen, methoxy, trifluoromethyl.

Y is tertiary N like =N or may be absent in cases where the X is directly attached to Z.

The bond between X and Z is single in cases where Y is absent and X is directly attached to Z

The bond between X and Y is double, when Y is present, and

Z═NH.

### BACKGROUND OF THE INVENTION

Tuberculosis (TB) is a life-threatening chronic infection worldwide and is primarily caused by the facultative intracellular bacterium Mycobacterium tuberculosis (Mtb). Mycobacterium tuberculosis is the pathogen responsible for TB which uses diverse strategies to survive in a variety of host injury and to evade immune systems. According to the World Health Organization (WHO), about one-third of the world's population is infected with Mtb, resulting in 9.4 million deaths from TB annually (WHO report 2010). Existing TB management regimen DOTS (direct observed therapy short-course) is a combination of three or four drugs, namely, isoniazid (INH), rifampicin (RMP), pyrazinamide (PZA), and ethambutol (EMB) or alternatively streptomycin (SM). This regimen requires to be taken by the patients throughout a 6-12 month period. The non-compliance by the patient and undesirable side effects associated with the long-term therapies lasting for 6-12 months are the two most significant factors responsible for the emergence of drug-resistant TB, namely, multidrug-resistant TB (MDR-TB) and extensively drug-resistant TB (XDR-TB). The MDR-TB is resistant to the most common first-line anti-TB drugs, i.e., INH and RMP, whereas XDR-TB is also resistant to the fluoroquinolones and at least one of the intravenous second-line drugs, i.e., kanamycin, capreomycin, or amikacin. According to the WHO report 2010, Of the 9.4 million TB cases in 2009, 66% were smear-positive and 11-13% were HIV positive, and the world's two most populous countries, India and China, account for more than 50% of the world's MDR-TB cases. The non-availability of a drug to successfully combat MDR-TB or XDR-TB emphasizes the urgent need for development of new therapeutics for tuberculosis (TB) with novel mechanisms of action to prevent the emergence of drug-resistant Mycobacterium tuberculosis (Mtb) strains, and to shorten the long-term therapies lasting for 6-12 months period.

The antiprotozoal drug Nitazoxanide (NTZ), which have already been approved by the U.S. Food and Drug Administration (FDA) in 2002, has also been recently reported to effectively kill both the replicating and nonreplicating Mtb at MIC value of 52.12 μM (16 μg/mL) and 60.38 μM (16 μg/mL) respectively with an excellent ability to evade wide spectrum Mtb resistance (Nathan et al, J. Med. Chem. 2009, 52, 5789). The ability of this drug to bypass the development of Mtb resistance, suggests it as novel lead compound in the area of tuberculosis research. Furthermore, it has been found that the anti-TB effect of NTZ was a time-dependent effect, which indicates that the killing of Mtb may be limited by slow penetration of the compound or by a subsequent activation step that may generate the final active species, as observed with INH, PA-824, and other anti-TB agents (Nathan et al, J. Med. Chem. 2009, 52, 5789). Therefore, further incorporation or substitution of lipophilic cores in this lead compound may furnish more potent candidate compound for the treatment of tuberculosis.

In view of above and in continuation of our previous research efforts toward the discovery of potent anti-TB agents, we describe in the present invention, the design, synthesis and in vitro screening of novel substituted 4-arylthiazoles as potent growth inhibitors of Mycobacterium tuberculosis.

### OBJECTS OF THE INVENTION

The main object of the present invention is to provide novel substituted 4-arylthiazoles that exhibit better therapeutic efficacy to treat tubercular infections.

Another objective, of the invention is to provide a process for preparation of substituted 4-arylthiazoles of formula A.

One more object of the present invention is to provide molecules useful for the treatment or prevention of tubercular infections caused by Mycobacterium species.

### SUMMARY OF THE INVENTION

The present invention relates to novel substituted 4-arylthiazoles, their preparation, and to their use as therapeutic agents, particularly in the prevention or treatment of tuberculosis. Accordingly, the present invention provides the compound of general formula A,

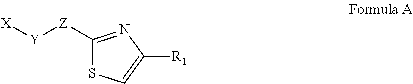

Wherein;
   R₁ is substituted/unsubstituted aryl or heteroaryl group of the structure

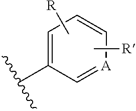

wherein,
A is CH or N

R and R′ are groups, which may be identical or different, selected from the group consisting of hydrogen, halogen, nitro, and methoxy
X is a group selected from the group consisting of

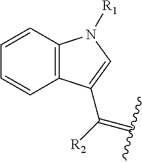

wherein, R₁ is a group selected from the group of benzyl and 2,4-dichlorobenzyl, while R2 is a group selected from hydrogen and methyl.

or, a group of structure

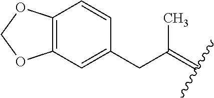 [SMILES: *=C(C)Cc1ccc2c(c1)OCO2]

or, a group of structure

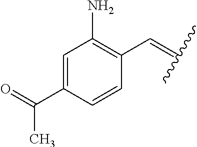 [SMILES: *=Cc1ccc(C(C)=O)cc1N]

or, a group of structure

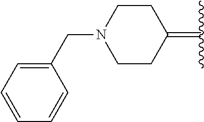 [SMILES: *=C1CCN(Cc2ccccc2)CC1]

or a group of structure

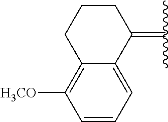 [SMILES: *=C1CCCc2c(OC)cccc21]

or a group of structure

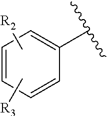

wherein, R2 and R3 may be same or different selected from the group of hydrogen, halogen, methoxy, trifluoromethyl.
Y is tertiary N like =N or may be absent in cases where the X is directly attached to Z
The bond between X and Z is single in cases where Y is absent and X is directly attached to Z
The bond between X and Y is double, when Y is present
Z=is NH.

In one embodiment of the present invention, the chemical formula of the representative compounds comprising:

4-(3,4-dimethoxyphenyl)-2-(2-(5-methoxy-3,4-dihydronaphthalen-1(2H)-ylidene)hydrazinyl)-thiazole (1a)

4-(2,4-dichlorophenyl)-2-(2-(5-methoxy-3,4-dihydronaphthalen-1(2H)-ylidene)hydrazinyl)-thiazole (1b)

4-(4-fluorophenyl)-2-(2-(5-methoxy-3,4-dihydronaphthalen-1(2H)-ylidene)hydrazinyl)-thiazole (1c)

2-(2-(5-methoxy-3,4-dihydronaphthalen-1(2H)-ylidene)hydrazinyl)-4-(3-nitrophenyl)-thiazole (1d)

2-(2-((1-(2,4-dichlorobenzyl)-1H-indol-3-yl)methylene)hydrazinyl)-4-(3,4-dimethoxyphenyl)-thiazole (2a)

2-(2-((1-(2,4-dichlorobenzyl)-1H-indol-3-yl)methylene)hydrazinyl)-4-(2,4-dichlorophenyl)-thiazole (2b)

2-(2-((1-(2,4-dichlorobenzyl)-1H-indol-3-yl)methylene)hydrazinyl)-4-(4-fluorophenyl)-thiazole (2c)
2-(2-(1-(1-benzyl-1H-indol-3-yl)ethylidene)hydrazinyl)-4-(3-chlorophenyl)-thiazole. (2d)
2-(2-(1-(1-benzyl-1H-indol-3-yl)ethylidene)hydrazinyl)-4-(4-fluorophenyl)-thiazole. (2e)
2-(2-(1-(1-benzyl-1H-indol-3-yl)ethylidene)hydrazinyl)-4-phenylthiazole. (2f)
2-(2-(1-(1-benzyl-1H-indol-3-yl)ethylidene)hydrazinyl)-4-(4-methoxyphenyl)-thiazole. (2g)

2-(2-(1-benzylpiperidin-4-ylidene)hydrazinyl)-4-(3-chlorophenyl)-thiazole. (3a)

2-(2-(1-benzylpiperidin-4-ylidene)hydrazinyl)-4-(4-fluorophenyl)-thiazole. (3b)

2-(2-(1-benzylpiperidin-4-ylidene)hydrazinyl)-4-(3-nitrophenyl)-thiazole. (3c)

2-(2-(1-benzylpiperidin-4-ylidene)hydrazinyl)-4-(4-methoxyphenyl)-thiazole. (3d)

2-(2-(1-benzylpiperidin-4-ylidene)hydrazinyl)-4-(pyridin-3-yl)-thiazole. (3e)

2-(2-(1-benzylpiperidin-4-ylidene)hydrazinyl)-4-phenylthiazole. (3f)

2-(2-(1-(benzo[d][1,3]dioxol-5-yl)propan-2-ylidene)hydrazinyl)-4-(3-nitrophenyl)-thiazole. (4a)

2-(2-(1-(benzo[d][1,3]dioxol-5-yl)propan-2-ylidene)hydrazinyl)-4-(3-chlorophenyl)-thiazole. (4b)

2-(2-(1-(benzo[d][1,3]dioxol-5-yl)propan-2-ylidene)hydrazinyl)-4-(4-methoxyphenyl)thiazole. (4c)

2-(2-(1-(benzo[d][1,3]dioxol-5-yl)propan-2-ylidene)hydrazinyl)-4-(4-fluorophenyl)-thiazole. (4d)

2-(2-(1-(benzo[d][1,3]dioxol-5-yl)propan-2-ylidene)hydrazinyl)-4-phenyl thiazole. (4e)

1-(3-amino-4-((2-(4-(3-chlorophenyl)thiazol-2-yl)hydrazono)methyl)phenyl)ethanone. (5a)

1-(3-amino-4-((2-(4-(4-methoxyphenyl)thiazol-2-yl)hydrazono)methyl)phenyl)ethanone. (5b)

1-(3-amino-4-((2-(4-(4-fluorophenyl)thiazol-2-yl)hydrazono)methyl)phenyl)ethanone. (5c)

1-(3-amino-4-((2-(4-(3-nitrophenyl)thiazol-2-yl)hydrazono)methyl)phenyl)ethanone. (5d)

1-(3-amino-4-((2-(4-phenyl thiazol-2-yl)hydrazono)methyl)phenyl)ethanone. (5e)

N,4-bis(4-fluorophenyl)thiazol-2-amine (6)

N-(2,5-dimethoxyphenyl)-4-(4-fluorophenyl)thiazol-2-amine (7a)

N-(2,5-dimethoxyphenyl)-4-(3-nitrophenyl)thiazol-2-amine (7b)

N-(2,5-dimethoxyphenyl)-4-(4-methoxyphenyl)thiazol-2-amine (7c)

4-(3,4-dimethoxyphenyl)-N-(2-(trifluoromethyl)phenyl)thiazol-2-amine (8a)
4-(4-fluorophenyl)-N-(2-(trifluoromethyl)phenyl)thiazol-2-amine (8b)
4-(3-nitrophenyl)-N-(2-(trifluoromethyl)phenyl)thiazol-2-amine (8c)
4-(4-methoxyphenyl)-N-(2-(trifluoromethyl)phenyl)thiazol-2-amine (8d)

In another embodiment of the invention wherein the structural formula of representative compounds of general formula A comprising:

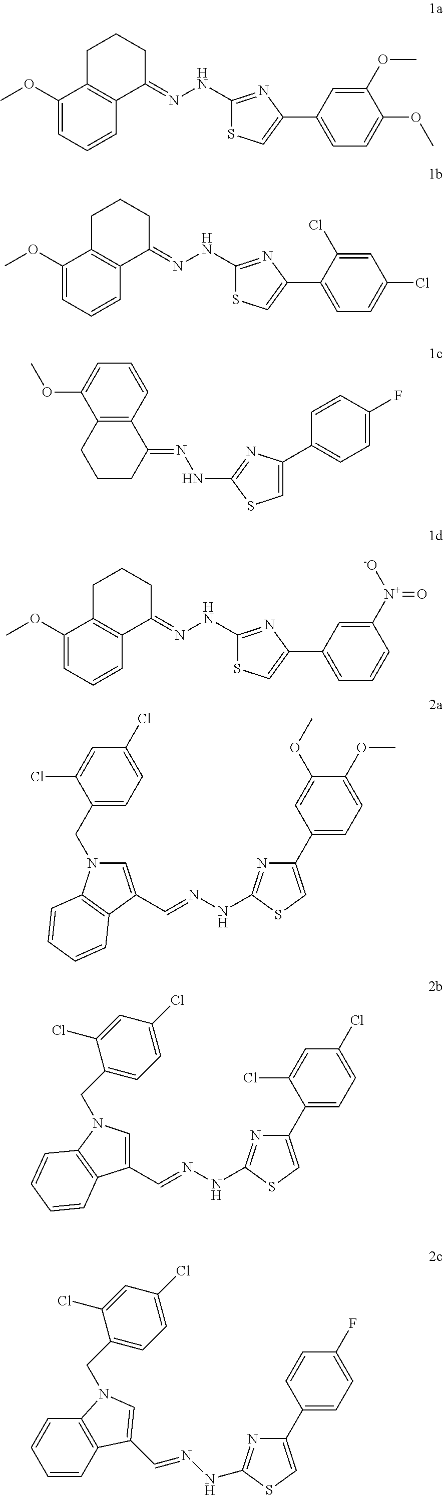

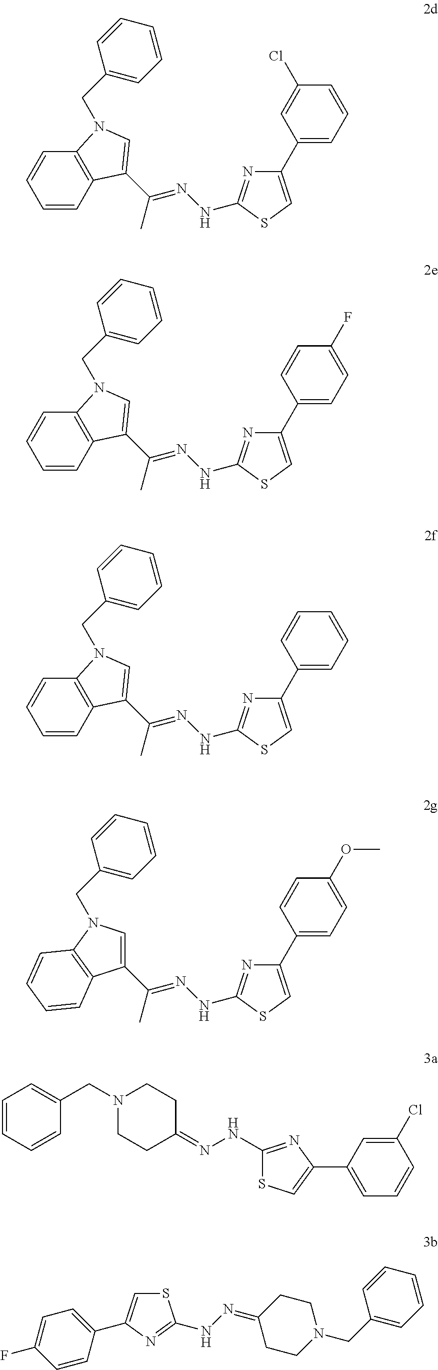

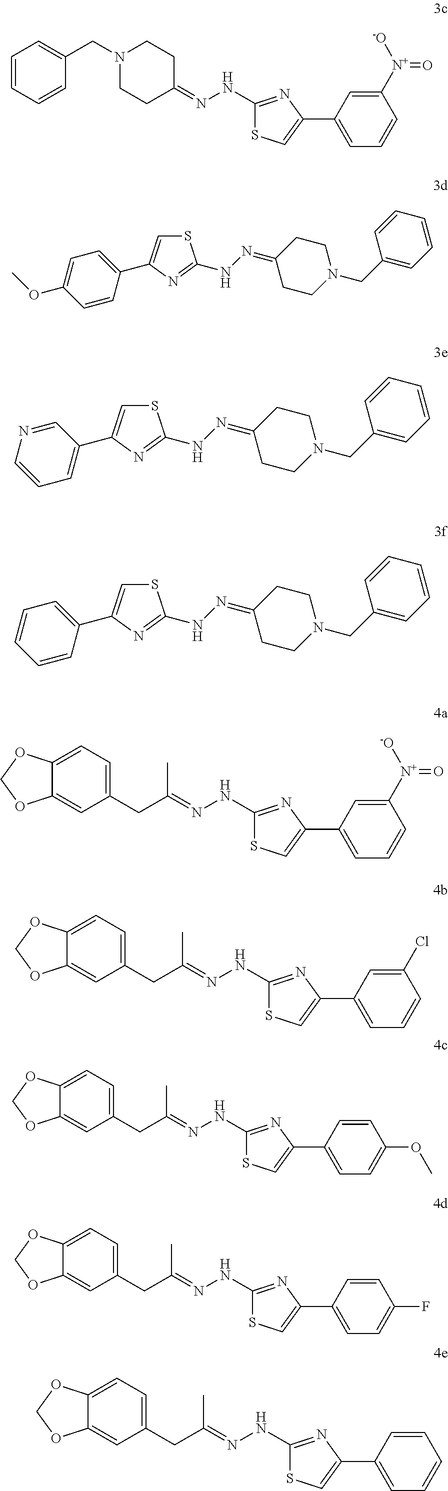

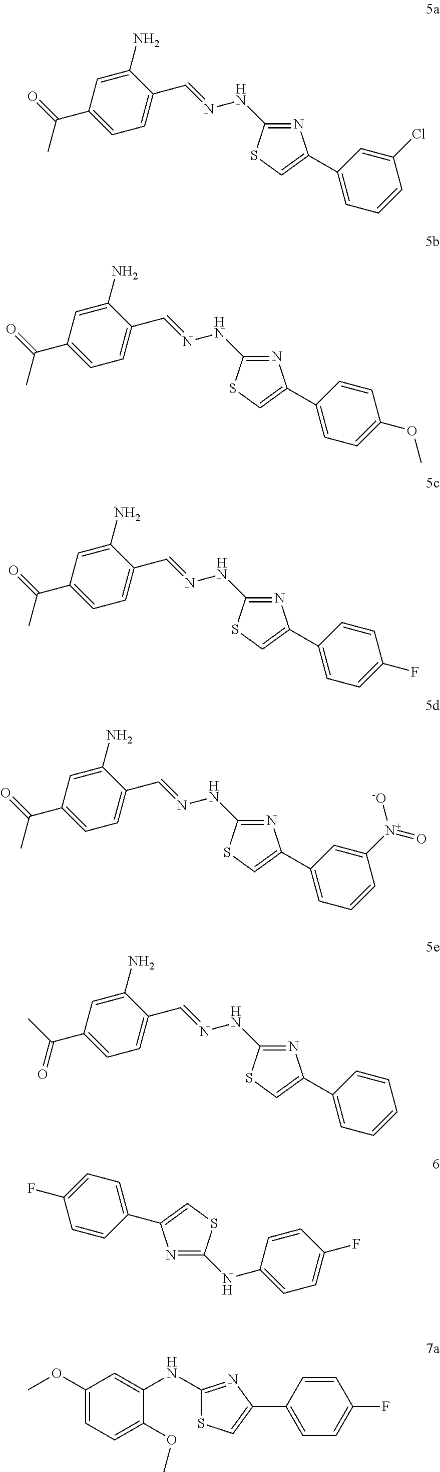

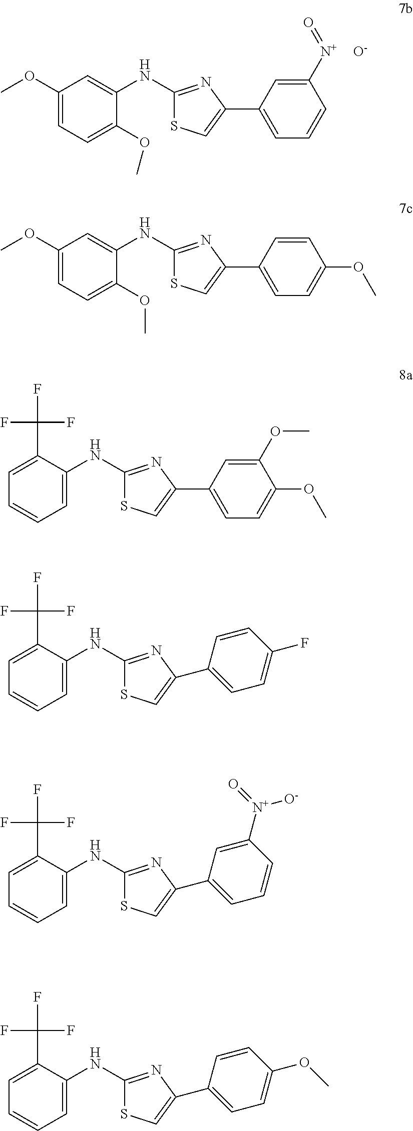

In still another embodiment of the present invention, the compound of general formula A are useful as anti-tuberculosis agent particularly in the treatment of multi-drug resistant tuberculosis (MDR-TB) and extensively drug resistant tuberculosis (XDR-TB).

In yet another embodiment of the present invention, the compound of the general formula A exhibiting MIC in the range of 6.25 to 32 μM causing 90% growth inhibition.

In still another embodiment of the present invention, the process for the synthesis of compounds of general formula A comprising the step:
reacting substituted/unsubstituted alpha bromoacetophenone with substituted hydrazine carbothioamide or substituted phenylthiourea in a solvent selected from a group consisting of anhydrous THF, acetone, ethanol, or other nonpolar/polar solvents at a temperature ranging between 10° C. to 60° C. for a period ranging between 0.5 hr to 24 hrs to provide compounds 1(a-d), 2(a-g), 3(a-f), 4(a-e), 5(a-e) or 6, 7(a-c), 8(a-d) respectively.

In yet another embodiment of the present invention, the alpha-bromoacetophenone is selected from a group consisting of 2-bromo-1-(3,4-dimethoxyphenyl)ethanone, 2-bromo-1-(2,4-dichlorophenyl)ethanone, 2-bromo-1-(4-fluorophenyl)ethanone, 2-bromo-1-(3-nitrophenyl)ethanone, 2-bromo-1-(3-chlorophenyl)ethanone, 2-bromo-1-phenylethanone, 2-bromo-1-(4-methoxyphenyl)ethanone, 2-bromo-1-(3-chlorophenyl)ethanone, 2-bromo-1-(4-fluorophenyl)ethanone, 2-bromo-1-(3-nitrophenyl)ethanone, 2-bromo-1-(4-methoxyphenyl)ethanone, 2-bromo-1-(pyridin-3-yl)ethanone, 2-bromo-1-phenylethanone, 2-bromo-1-(3-nitrophenyl)ethanone, 2-bromo-1-(3-chlorophenyl)ethanone, 2-bromo-1-(4-methoxyphenyl)ethanone, 2-bromo-1-(4-fluorophenyl)ethanone, 2-bromo-1-phenylethanone, 2-bromo-1-(3-chlorophenyl)ethanone, 2-bromo-1-(4-fluorophenyl)ethanone, 2-bromo-1-(3-nitrophenyl)ethanone, 2-bromo-1-(3,4-dimethoxyphenyl)ethanone, 2-bromo-1-(2,5-dimethoxyphenyl)ethanone, or 2-bromo-1-(4-fluorophenyl)ethanone

In still another embodiment of the present invention, the substituted Hydrazine carbothioamide is selected from the group consisting of 2-(5-methoxy-3,4-dihydronaphthalen-1(2H)-ylidene)hydrazinecarbothioamide, 2-((1-(2,4-dichlorobenzyl)-1H-indol-3-yl)methylene)hydrazine carbothioamide, 2-((1-benzyl-1H-indol-3-yl)methylene)hydrazinecarbothioamide, 2-(1-benzylpiperidin-4-ylidene)hydrazinecarbothioamide,-(1-(benzo[d][1,3]dioxol-5-yl)propan-2-ylidene)hydrazinecarbothioamide, or 2-(4-acetyl-2-aminobenzylidene)hydrazinecarbothioamide.

In yet another embodiment of the present invention, the substituted phenyl thiourea is selected from a group consisting of 1-(4-fluorophenyl)thiourea, 1-(3-nitrophenyl)thiourea, 1-(4-methoxyphenyl)thiourea, 1-(2-(trifluoromethyl)phenyl)thiourea, 1-(2-(trifluoromethyl)phenyl)thiourea, 1-(2-(trifluoromethyl)phenyl)thiourea, or 1-(2-(trifluoromethyl)phenyl)thiourea.

In still another embodiment of the present invention, the method of preparation of substituted phenyl thiourea comprising
a) reacting substituted aniline with benzoyl isothiocyanate in dry benzene for 8-10 hrs to afford the corresponding N-(substituted phenylcarbamothioyl)benzamide represented by formula B wherein R2 and R3 may be same or different selected from a group consisting of hydrogen, fluoro, methoxy, nitro, trifluoromethyl,

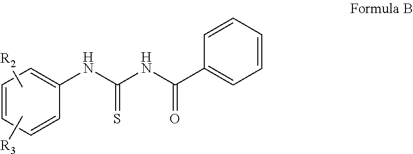

b) debenzoylating compounds of formula B as obtained in step (a) by refluxing in 10% NaOH aqueous solution at 100° C. for 1 hr to afford the corresponding phenylthiourea of formula C wherein R2 and R3 may be same or different selected from a group consisting of hydrogen, fluoro, methoxy, nitro, trifluoromethyl,

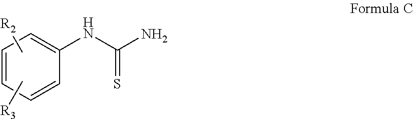

In still another embodiment, method for producing compounds of Formula A comprises:
reacting substituted/unsubstituted alpha-bromoacetophenone with substituted phenylthiourea represented by formula C wherein R2 and R3 may be same or different selected from a group consisting of hydrogen, fluoro, methoxy, nitro, trifluoromethyl, or substituted hydrazine carbothioamide represented by formula D wherein X is selected from groups as described above to define compound of General Formula A, in a solvent selected from a group

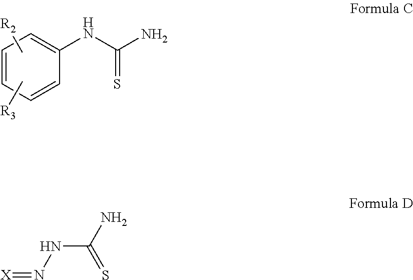

consisting of anhydrous THF, acetone, ethanol, or other nonpolar/polar solvents at a temperature ranging between 10° C. to 60° C. for a period ranging between 0.5 hr to 24 hrs to provide compounds of general formula A.

### BRIEF DESCRIPTION OF DRAWINGS

FIG. 1 outlines the rational design of modified analogues of nitazoxanide (NTZ).

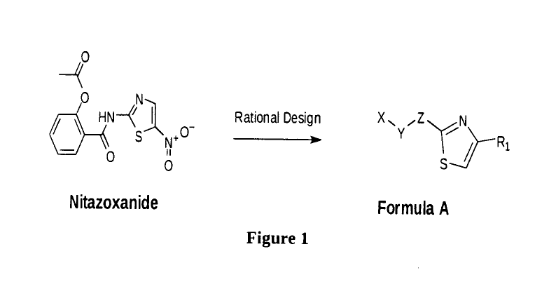

FIG. 2 outlines the scheme for the synthesis of compounds (1a-d, 2a-g, 3a-f, 4a-e, 5a-e) of formula A.

FIG. 3 outlines the synthesis of compounds (6, 7a-c, 8a-d) of formula A

FIG. 4 represents a plot between the percentage Vero cell growth inhibitory activities and the concentration of these two active compounds 1a and 1c.

FIG. 5 represents in vitro anti-TB growth index (GI), against M. tuberculosis H₃₇Rv plotted against number of days (day 1 to day 11) for compounds 1a (A), 1c (B) and 6 (C).

### ABBREVIATIONS

AcOH: acetic acid; ADA: agar dilution assay; CFU: colony-forming unit; DMEM: Dulbecco's minimal essential medium; DOTS: direct observed therapy short-course; EMB: ethambutol; EtOAc: ethyl acetate, FBS: fetal bovine serum; GI: growth index; INH: isoniazid; IR: infra-red spectroscopy; LTBI: latent TB infection; MDR-TB: multi-drug resistant tuberculosis; MIC: minimum inhibitory concentrations; NMR: nuclear magnetic resonance, MS: mass spectroscopy; PZA: pyrazinamide; RMP: rifampicin; SI: selectivity index; SM: streptomycin; TB: tuberculosis; THF: tetrahydrofuran; TLC: thin layer chromatographic; WHO: world health organization; XDR-TB: extensively-drug resistant tuberculosis.

### DETAILED DESCRIPTION OF THE INVENTION

The present invention relates to novel substituted 4-arylthiazoles, their preparation, and to their use as therapeutic agents, particularly in the prevention or treatment of tuberculosis. The present invention particularly relates to compounds of formula A:

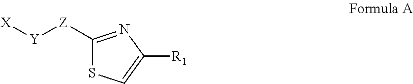

wherein:
R₁ is substituted/unsubstituted aryl or heteroaryl group of the structure

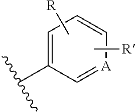

wherein, A may be CH or N
R and R′ are groups, which may be identical or different, selected from the group consisting of hydrogen, halogen, nitro, amino, and methoxy.
X is a group selected from the group consisting of

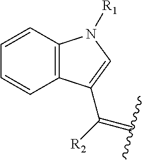

wherein, R₁ is a group selected from the group of benzyl and 2,4-dichlorobenzyl, while R2 is a group selected from hydrogen and methyl.

or, a group of structure

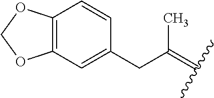 [SMILES: *=C(C)Cc1ccc2c(c1)OCO2]

or, a group of structure

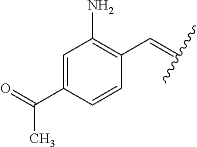 [SMILES: *=Cc1ccc(C(C)=O)cc1N]

or, a group of structure

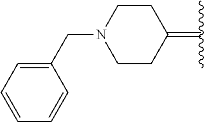 [SMILES: *=C1CCN(Cc2ccccc2)CC1]

or a group of structure

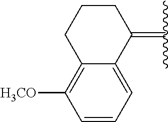 [SMILES: *=C1CCCc2c(OC)cccc21]

or a group of structure

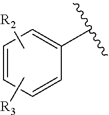

wherein, R2 and R3 may be same or different selected from the group of hydrogen, halogen, methoxy, trifluoromethyl.
Y is tertiary N like =N or may be absent in cases where the X is directly attached to Z.
The bond between X and Z is single in cases where Y is absent and X is directly attached to Z.
The bond between X and Y is double, when Y is present.
Z=is NH.

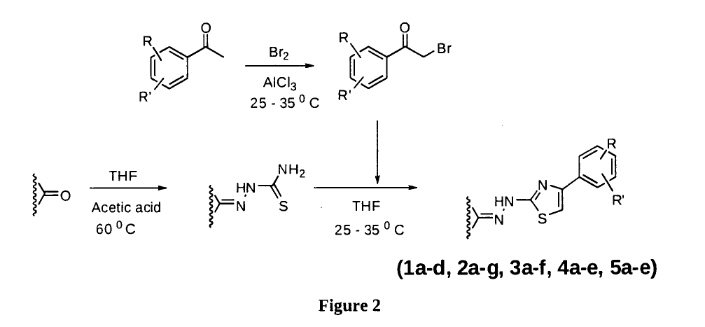

FIG. 2 outlines the synthesis of the compounds (1a-d, 2a-g, 3a-f, 4a-e, 5a-e) of formula A. The thiosemicarbazone was obtained by the reaction between the ketones and thiosemicarbazide in absolute ethanol with catalytic amount of acetic acid at 80° C. The reaction of thiosemicarbazone with different freshly prepared α-bromo-acetophenones in THF as solvent afforded the corresponding 4-arylthiazol-2-yl hydrazones (1a-d, 2a-g, 3a-f, 4a-e, 5a-e) in high yields (80-90%).

The different substituted α-bromoacetophenones were synthesized using bromine (Br₂) in the presence of aluminium chloride (AlCl₃) as catalyst and dry ether or THF as solvent.

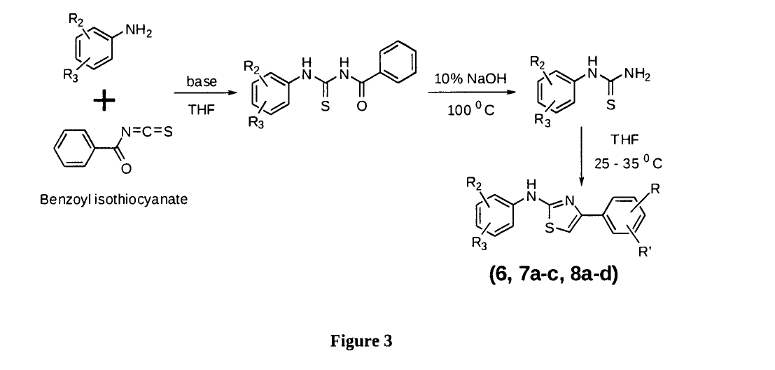

The FIG. 3 outlines the synthesis of compounds (6, 7a-c, 8a-d). The reaction of substituted aniline with benzoyl isothiocyanate in ethanol affords the corresponding N-(substituted phenylcarbamothioyl)benzamide intermediate, which upon debenzoylation reaction yields the corresponding phenylthiourea. The condensation of the (un)substituted phenylthiourea with different freshly synthesized a-bromo-acetophenones affords the corresponding final compounds (6, 7a-c, 8a-d).

Further details of the preparation of the compounds of formula-A are given in examples.

Biological Evaluation

Agar Dilution Assay: Compounds were dissolved in dimethylsulfoxide (DMSO) to make 5 mg/mL stock solutions. Serial dilutions from stocks were also made in DMSO. Standard anti-TB drugs (isoniazid and rifampicin) were used as positive control and the vehicle (DMSO) was used as negative control. An amount of 0.1 mL of serially diluted test compounds or standard drugs were added to 1.9 mL Middlebrook 7H10 agar medium (with OADC supplement, in glass tubes). 0.1 mL/tube DMSO was used as vehicle control. The contents were mixed and allowed to solidify as slants. Three-week old culture of M. tuberculosis H37Rv was harvested from Lowenstein-Jensen medium and its suspension (1 mg/mL, equivalent to 10⁸ bacilli) was made in normal saline containing 0.05% Tween-80. 10 μL of 1:10 dilution of this suspension (−10⁵ bacilli) was inoculated into each tube and incubated at 37° C. for 4 weeks. The lowest concentration of the compound up to which there was no visible growth of bacilli was its minimum inhibitory concentration (MIC).

BACTEC Assay: The in vitro anti-TB activity of compounds was measured by BACTEC-460TB radiometric method for determination of the minimum inhibitory concentration (MIC). The M. tuberculosis H37Rv strain was used to grow in 7H12 medium containing ¹⁴C labeled palmitic acid as substrate during which ¹⁴CO₂was liberated. The amount of ¹⁴CO₂ detected by the BACTEC system reflected the growth of the organism and was expressed in terms of the “Growth Index” (GI). By adding compound to the medium, suppression of growth of the M. tuberculosis was detected by decline of the daily GI as compared to the control, if compound is active at that concentration.

Cytotoxicity Studies: The compounds for cytotoxicity were tested in an in vitro model for toxicity with Vero monkey kidney cells using Resazurin assay. The Vero cells (ATCC CCL-8 1) were seeded overnight at 1×10⁴−3×10⁶ cells per well in 96-well plates at 37° C. in RPMI supplemented with 10% heat-inactivated fetal bovine serum and 5% CO₂. Cells were exposed to dilutions of experimental and control drugs in triplicate for 2 h with each compound at a range of concentrations from 100-1.56 μg/ml. Rifampicin was used as a control at the same concentrations. Each well had 100 μL of the test material in serially descending concentrations. After 72 h of incubation, 10 μL of Resazurin indicator solution (0.1%) was added and incubation was continued for 4-5 h. Any color change from purple to pink or colorless was recorded as positive. Fluorescence was measured of each sample with excitation wavelength at 530 nm and emission wavelength at 590 nm using the BMG Polar Star Galaxy. The CC₅₀ values (50% inhibitory concentrations) were calculated by plotting fluorescence values using Microsoft excel template. The data from this toxicity testing and MIC values were used to calculate a selectivity index (SI), the ratio of CC₅₀: MIC.

Among the synthesized compounds, six compounds, namely 1a-d, 2b and 6 effectively inhibited the growth of replicating Mtb over a long period (4 weeks) with micromolar MIC values. Structurally, all the above six active compounds except 1d are non-nitro compounds unlike NTZ and still have 2-3 times better anti-TB activity than the drug NTZ. Therefore, above data suggests that the presence of nitro group is not mandatory for potential inhibition of the growth of Mtb and hence, for promising anti-TB activity. Also, looking at the structure of NTZ, TIZ and identified active compounds (1a-d, 2b and 6), it is also evident that the presence of a nitro group at the position 5 of the aminothiazole is not essential for anti-TB activity, although it may be necessary for anti-protozoal activity.

TABLE 1
Structures and in vitro anti-TB activity of synthesized and
reference compounds from agar dilution assay.

| | | Mtb | | In vitro | |
|---|---|---|---|---|---|
| | | H37Rv | | cytotoxicity | |
| | | MIC | | Vero cells | |
| S.N. | Compd | μM | μg/mL | CC₅₀^{a} (μM) | SI^{b} |
| 1 | 1a | 15.28 | 6.25 | >244 | ≧16 |
| 2 | 1b | 29.90 | 12.50 | ND^{c} | ND |
| 3 | 1c | 17.03 | 6.25 | >300 | ≧17 |
| 4 | 1d | 31.73 | 12.50 | ND | ND |
| 5 | 2a | | >12.50 | ND | ND |
| 6 | 2b | 22.89 | 12.50 | ND | ND |
| 7 | 2c | | >12.50 | ND | ND |
| 8 | 6 | 6.25 | 1.80 | ND | ND |
| 9 | 7a | | >12.50 | ND | ND |
| 10 | 7b | | >12.50 | ND | ND |
| 11 | 7c | | >12.50 | ND | ND |
| 12 | 8a | | >12.50 | ND | ND |
| 13 | 8b | | >12.50 | ND | ND |
| 14 | 8c | | >12.50 | ND | ND |
| 15 | 8d | | >12.50 | ND | ND |
| | Isoniazid (INH) | ND | <1 | ND | ND |
| | Rifampicin (RMP) | 0.24 | 0.20 | ND | ND |
| | Nitazoxanide (NTZ) | 52.12 | 16.00 | ND | ND |
| | Tizaxonide (TIZ) | 60.38 | 16.00 | ND | ND |

^{a}in vitro cytotoxicity with Vero monkey kidney cells using resazurin assay;
^{b}selectivity index;
^{c}not determined

TABLE 2
Structures and in vitro anti-TB activity of
synthesized and reference compounds.

| | Mtb H37Rv MIC (μM) | | |
|---|---|---|---|
| Compd | ADA^{a} | BACTEC^{b} | Vero cells CC₅₀ (μM) |
| 1a | 15.28 | 6.25 | >244 |
| 1c | 17.03 | 6.25 | >300 |
| 6 | 6.25 | 12.5 | ND |
| Streptomycin (SM) | ND^{c} | 6.00 | ND |
| Nitazoxanide (NTZ) | 52.12 | ND | ND |

^{a}agar dilution assay;
^{b}BACTEC-460B radiometric assay;
^{c}not determined

In comparison to the observed anti-TB MIC value of 52.12 [symbol] μM (16 μg/mL) for the drug NTZ, the compound 1a, comprising (5-methoxy-3, 4-dihydronaphthalen-1(2H)-ylidene)hydrazine and 3, 4-dimethoxyphenyl groups linked to position 2 and 4 of thiazole respectively, exhibited about 3 times better anti-TB activity with MIC value of 15.28 μM in agar dilution assay. In the same series, another compound 1c, bearing 4-fuorophenyl group at position 4 of thiazole, also exhibited about 3 times better Mtb growth inhibitory activity with MIC value of 17.03 μM than the drug NTZ. Another two compounds 1b and 1d in the same series, bearing 2,4-dicholoro and 3-nitrophenyl groups respectively at position 4 of thiazole, also exhibited good Mtb growth inhibitory activity with MIC of 29.90 and 31.73 μM respectively. Although, these two compounds were less potent than compounds 1a and 1c, they were still about 2 times more potent than the drug NTZ (MIC=52.12 μM) toward the growth inhibition of replicating Mtb H₃₇Rv.

The compound 2b, having 1-(2, 4-dichlorobenzyl)-3-(hydrazonomethyl)-1H-indole and 2,4-dichlorophenyl group linked to the position 2 and 4 respectively, did not show any improved anti-TB activity (MIC=22.89 μM) over compounds 1a (MIC=15.28 μM) and is (MIC=17.03 μM). However, these compounds were about 3 times more potent than the drug NTZ. Other compounds (2a and 2c) in this series had anti-TB MIC value of >12.5 μg/mL. Interestingly, among all the synthesized compounds, compound 6 was the most potent Mtb growth inhibitor (MIC=6.25 μM) in agar dilution assay and thus, was about 9 times more potent than the drug NTZ (MIC=52.12 μM).

In order to further confirm the Mtb growth inhibitory potential of the above three compounds, namely 1a, 1c, and 6 with 3-9 times superior anti-TB activity over the drug NTZ in agar dilution assay, these compounds were further subjected to another widely used assay system referred to as BACTEC 460TB radiometric method (BACTEC assay). In this assay, streptomycin (SM) was used as positive control. The observed anti-TB activities of these three compounds and SM are summarized in Table 2.

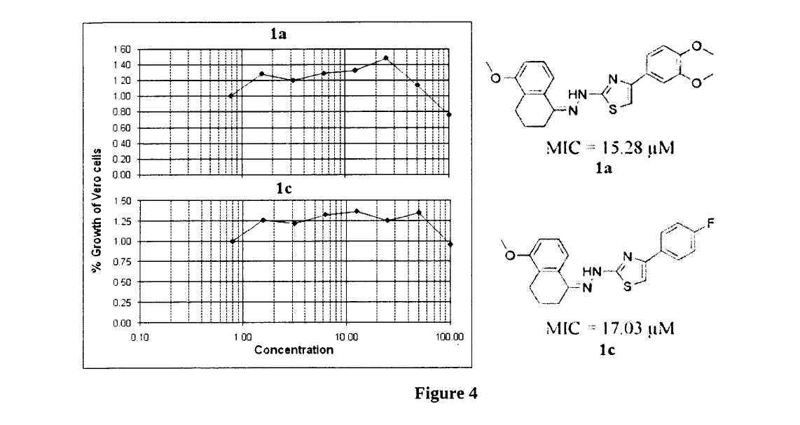

In the BACTEC assay, two compounds 1a and 1c again effectively inhibited the growth of Mtb with MIC value of 6.25 μM each. However, the most active compound 6 (MIC=6.25 μM in agar dilution assay) exhibited relatively poor anti-TB activity (MIC=12.5 μM) in the BACTEC assay. Finally, Vero monkey kidney cells were used for the in vitro toxicity evaluation of the active compounds 1a and 1c, using Resazurin assay. FIG. 4 shows the plot between the percentage Vero cell growth inhibitory activities and the concentration of these two active compounds. These compounds failed to show any cytotoxicity (CC₅₀>244 μM), thus suggesting that their anti-TB activity was not due to some general cytotoxicity. Furthermore, these two compounds 1a and 1c had high selectivity index (SI) of ≧39 and ≧48 respectively.

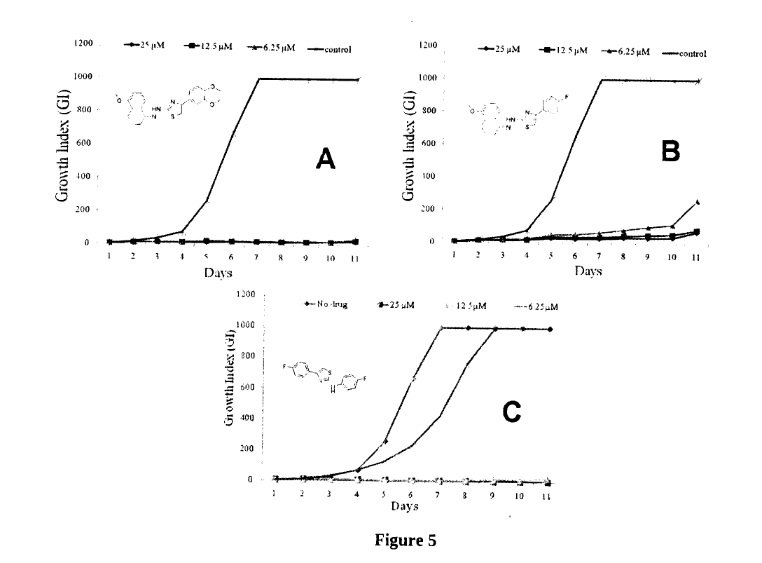

FIG. 5 shows in vitro anti-TB growth index (GI), against M. tuberculosis H₃₇Rv plotted against number of days (day 1 to day 11) for compounds 1a (A), 1c (B) and 6 (C). The compound 1a was very effective for inhibiting the Mtb growth till day 11 at 6.25 μM, while the compound 1c was comparatively lesser effective on day 10 onwards at 6.25 μM in BACTEC assay as shown in FIG. 5. It is evident from the growth index (GI) graphs plotted for compounds 1a and 1c that the administration (6.25 μM) of the compound 1a on day 1 was sufficient to completely inhibit the growth of the mycobacterium till day 10, thus, reflecting on the interesting bactericidal nature of this compound. However, the other active compound 1c could not retain the complete inhibition till day 10. Nevertheless, it did show potent inhibition of the growth of replicating Mtb H₃₇Rv. The relatively less potent compound 6 was found to effectively inhibit Mtb growth till day 11 at ≧12.5 μM. The plot (FIG. 5) of growth index vs days is provided as supplementary material.

Based on the above Mtb growth inhibitory activity data from the two distinct assay systems and cytotoxicity data from Resazurin assay, the two compounds, namely 1a and 1c, were selected as novel lead compounds with 3 times better anti-TB activity over the drug NTZ. These two compounds belong to similar prototype with a bulkier 5-methoxy-3,4-dihydronaphthalen-1(2H)-imine on one end and a substituted phenyl ring on the other end of 2-aminothiazole core. In addition, these two compounds are non-nitro compounds and thus, reflect the lesser importance of the nitro group for potential anti-TB activity.

### THE FOLLOWING EXAMPLES ARE GIVEN BY WAY OF ILLUSTRATION AND SHOULD NOT BE CONSTRUED TO LIMIT THE SCOPE OF THE INVENTION

General Procedure for the Synthesis of Substituted α-Bromoacetophenones

To a solution of (un)substituted acetophenones (1M) in dry ether with a catalytic amount of AlCl₃, was added bromine (2.5M), were stirred for 1 hr at 0° C. and then allowed to warm at room temperature until the disappearance of HBr. The white solid was filtered and washed with dry ether to give pure product in good yield (76-80%).

General Procedure for the Syntheses of Thiosemicabazone

A mixture of ketone (1M) and thiosemicarbazide (1.2M) in THF with a catalytic amount of acetic acid was stirred for about 10 hrs at 80° C. The reaction mixture was cooled to room temperature and then evaporated under vacuum to yield the desired thiosemicarbazone as solid, which was washed with diethyl ether and dried.

2-(5-methoxy-3,4-dihydronaphthalen-1(2H)-ylidene)hydrazinecarbothioamide: % yield=78%. m.p. 175° C., MS: m/z 251 (M+1)⁺.

2-((1-(2,4-dichlorobenzyl)-1H-indol-3-yl)methylene)hydrazinecarbothioamide: % yield=76%. m.p. 156° C., MS: m/z 378 (M+1)⁺.

2-(1-benzylpiperidin-4-ylidene)hydrazinecarbothioamide: % yield=78%. m.p. 138° C., MS: m/z 263 (M+1)⁺.

2-(1-(1-benzyl-1H-indol-3-yl)ethylidene)hydrazinecarbothioamide: % yield=76%. m.p. 214° C., MS: m/z 323 (M+1)⁺.

2-(1-(benzo[d][1,3]dioxol-5-yl)propan-2-ylidene)hydrazinecarbothioamide: % yield=76%. m.p. 218° C., MS: m/z 252 (M+1)⁺.

### Example 1

### 4-(3,4-dimethoxyphenyl)-2-(2-(5-methoxy-3,4-dihydronaphthalen-1(2H)-ylidene)hydrazinyl)-thiazole (1a)

Method A: The reaction of the 2-(5-methoxy-3,4-dihydronaphthalen-1(2H)-ylidene)hydrazinecarbothioamide (249 mg, 1 mM) and freshly synthesized 2-bromo-1-(3,4-dimethoxyphenyl)ethanone (259 mg, 1 mM) in anhydrous THF at 28° C. for 30 mins resulted in the formation of suspension, which was filtered and dried to yield the final product (75% yield).

mp 200° C. ¹H NMR (300 MHz, CDCl₃): δ (ppm) 7.34 (d, 1H, J=7.62 Hz), 7.28 (d, 1H, J=7.98 Hz), 7.25 (dd, 1H, J=1.82 Hz, 1.91 Hz), 7.23 (d, 1H, J=6.23 Hz), 7.11(t, 1H, J=8.13 Hz, 7.86 Hz), 7.01 (s, 1H), 6.98 (t, 1H, J=7.86 Hz, 7.21 Hz), 2.76 (t, 2H, J=5.23 Hz, 5.19 Hz), 2.11 (m, 2H), 1.64(t, 2H, J=5.94 Hz, 5.79 Hz), 3.82 (s, 9H). IR (KBr) cm⁻¹: 3474, 3072, 2942, 2368, 1613, 1263,765. MS: m/z 410 (M+1)⁺. HRMS (ESI) m/z [M+1]⁺ calcd for C₂₂H₂₃N₃O₃S: 410.1460; found: 410.1716.

Method B: To the solution of 2-(5-methoxy-3,4-dihydronaphthalen-1(2H)-ylidene)-hydrazinecarbothioamide (249 mg, 1 mM) in anhydrous acetone at 30° C., was added freshly synthesized 2-bromo-1-(3,4-dimethoxyphenyl)ethanone (259 mg, 1 mM) and stirred for 4 hrs. The completion of the reaction was monitored using TLC method. After the completion of the reaction, the solvent was evaporated under vacuum, and then treated with ether to yield suspension, which was filtered and dried to yield the final product (56% yield).

Method C: A mixture of equimolar quantities of 2-(5-methoxy-3,4-dihydronaphthalen-1(2H)-ylidene)hydrazinecarbothioamide (249 mg, 1 mM) and freshly synthesized 2-bromo-1-(3,4-dimethoxyphenyl)ethanone (259 mg, 1 mM) in ethanol was refluxed at 60° C. for 6 hrs. The progress of reaction was monitored by TLC at appropriate time interval. The solution was poured on to the crushed ice and the precipitated solid collected by filtration, suspended in water and neutralized with NaHCO₃ to get the product. The product was recrystallized from ether to yield the pure product. (62% yield)

### Example 2

### 4-(2,4-dichlorophenyl)-2-(2-(5-methoxy-3,4-dihydronaphthalen-1(2H)-ylidene)hydrazinyl)thiazole (1b)

The reaction of the 2-(5-methoxy-3,4-dihydronaphthalen-1(2H)-ylidene)hydrazine carbothioamide (249 mg, 1 mM) and freshly synthesized 2-bromo-1-(2,4-dichlorophenyl)ethanone (268 mg, 1 mM) in anhydrous THF at 27° C. for 90 mins resulted in the formation of suspension, which was filtered and dried to yield the final product (78% yield). mp 200° C. MS: m/z 418 (M+1)⁺.

### Example 3

### 4-(4-fluorophenyl)-2-(2-(5-methoxy-3,4-dihydronaphthalen-1(2H)-ylidene)hydrazinyl)thiazole (1c)

The reaction of the 2-(5-methoxy-3,4-dihydronaphthalen-1(2H)-ylidene)hydrazine carbothioamide (249 mg, 1 mM) and freshly synthesized 2-bromo-1-(4-fluorophenyl)ethanone (217 mg, 1 mM) in anhydrous THF at 32° C. for 45 mins resulted in the formation of suspension, which was filtered and dried to yield the final product (73% yield). mp 198° C. MS: m/z 368 (M+1)⁺.

### Example 4

### 2-(2-(5-methoxy-3,4-dihydronaphthalen-1(2H)-ylidene)hydrazinyl)-4-(3-nitrophenyl)thiazole (1d)

The reaction of the 2-(5-methoxy-3,4-dihydronaphthalen-1(2H)-ylidene)hydrazine carbothioamide (249 mg, 1 mM) and freshly synthesized 2-bromo-1-(3-nitrophenyl)ethanone (244 mg, 1 mM) in anhydrous THF at 29° C. for 30 mins resulted in the formation of suspension, which was filtered and dried to yield the final product (80% yield). mp 185° C. MS: m/z 395 (M+1)⁺.

### Example 5

### 2-(2-((1-(2,4-dichlorobenzyl)-1H-indol-3-yl)methylene)hydrazinyl)-4-(3,4-dimethoxyphenyl)thiazole (2a)

The reaction of the 2-((1-(2,4-dichlorobenzyl)-1H-indol-3-yl)methylene)hydrazine carbothioamide (377 mg, 1 mM) and freshly synthesized 2-bromo-1-(3,4-dimethoxyphenyl)ethanone (259 mg. 1 mM) in anhydrous THF at 32° C. for 1 hr resulted in the formation of suspension, which was filtered and dried to yield the final product (76% yield).

mp 205° C. ¹H NMR (300 MHz, CDCl₃): δ (ppm) 8.71 (s, 1H), 8.23 (d, 1H, J=6.90 Hz), 8.13 (t, 1H, J=7.95 Hz, 7.83 Hz), 8.04 (s, 1H), 7.78 (d, 1H, J=6.36 Hz), 7.59 (s, 2H), 7.47 (d, 2H, J=10.61 Hz), 7.42 (d, 2H, J=3.90 Hz), 6.89 (d, 1H, J=8.79 Hz), 5.56 (s, 2H), 2.50 (s, 6H). IR (KBr) cm⁻¹: 3375, 3087, 2933, 2368, 1626, 1259, 738. MS: m/z 537 (M+1)⁺. HRMS (ESI) m/z [M+1]⁺ calcd for C₂₇H₂₂Cl₂N₄O₂S 537.0841; found: 537.0889.

### Example 6

### 2-(2-((1-(2,4-dichlorobenzyl)-1H-indol-3-yl)methylene)hydrazinyl)-4-(2,4-dichlorophenyl)thiazole (2b)

The reaction of the 2-((1-(2,4-dichlorobenzyl)-1H-indol-3-yl)methylene)hydrazine carbothioamide (377 mg, 1 mM) and freshly synthesized 2-bromo-1-(2,4-dichlorophenyl)ethanone (268 mg, 1 mM) in anhydrous THF at 30° C. for 45 mins resulted in the formation of suspension, which was filtered and dried to yield the final product (79% yield).

mp 185° C. MS: m/z 547 (M+1)⁺.

### Example 7

### 2-(2-((1-(2,4-dichlorobenzyl)-1H-indol-3-yl)methylene)hydrazinyl)-4-(4-fluorophenyl)thiazole (2c)

The reaction of the 2-((1-(2,4-dichlorobenzyl)-1H-indol-3-yl)methylene)hydrazine carbothioamide (377 mg, 1 mM) and freshly synthesized 2-bromo-1-(4-fluorophenyl)ethanone (217 mg, 1 mM) in anhydrous THF at 30° C. for 90 mins resulted in the formation of suspension, which was filtered and dried to yield the final product (78% yield). mp 198° C. MS: m/z 496 (M+1)⁺.

### Example 8

### 2-(2-(1-(1-benzyl-1H-indol-3-yl)ethylidene)hydrazinyl)-4-(3-chlorophenyl)thiazole (2d)

The reaction of the 2-((1-benzyl-1H-indol-3-yl)methylene)hydrazinecarbothioamide (308 mg, 1 mM) and freshly synthesized 2-bromo-1-(3-chlorophenyl)ethanone (234 mg, 1 mM) in anhydrous THF at 32° C. for 60 mins resulted in the formation of suspension, which was filtered and dried to yield the final product (76% yield). MS: m/z 457 (M+1)⁺. ¹H NMR (300 MHz, CDCl₃): δ (ppm) 8.23 (m, 2H), 7.92 (s, 1H), 7.88 (d, 2H, J=5.1 Hz), 7.69 (d, 2H, J=1.98 Hz), 7.35 (t, 2H, J=7.44 Hz, 2.07 Hz), 7.25 (t, 2H, J=2.55 Hz, 6.63 Hz), 5.50 (s, 2H). IR (KBr) cm⁻¹: 3408, 3053, 2863, 2366, 1621, 1231, 773. MS: m/z 457 (M+1)⁺. HRMS (ESI) m/z [M+1]⁺ calcd for C₂₅H₁₇Cl₂FN₄S: 457.1175; found: 457.1222.

### Example 9

### 2-(2-(1-(1-benzyl-1H-indol-3-yl)ethylidene)hydrazinyl)-4-(4-fluorophenyl)thiazole (2e)

The reaction of the 2-((1-benzyl-1H-indol-3-yl)methylene)hydrazinecarbothioamide (308 mg, 1 mM) and freshly synthesized 2-bromo-1-(4-fluorophenyl)ethanone (217 mg, 1 mM) in anhydrous THF at 35° C. for 60 mins resulted in the formation of suspension, which was filtered and dried to yield the final product (81% yield). MS: m/z 441 (M+1)⁺.

### Example 10

### 2-(2-(1-(1-benzyl-1H-indol-3-yl)ethylidene)hydrazinyl)-4-phenyl thiazole (2f)

The reaction of the 2((1-benzyl-1H-indol-3-yl)methylene)hydrazinecarbothioamide (308 mg, 1 mM) and freshly synthesized 2-bromo-1-phenylethanone (200 mg, 1 mM) in anhydrous THF at 34° C. for 90 mins resulted in the formation of suspension, which was filtered and dried to yield the final product (75% yield). MS: m/z 423 (M+1)⁺.

### Example 11

### 2-(2-(1-(1-benzyl-1H-indol-3-yl)ethylidene)hydrazinyl)-4-(4-methoxyphenyl)-thiazole (2g)

The reaction of the 2-((1-benzyl-1H-indol-3-yl)methylene)hydrazinecarbothioamide (308 mg, 1 mM) and freshly synthesized 2-bromo-1-(4-methoxyphenyl)ethanone (229 mg, 1 mM) in anhydrous THF at 32° C. for 45 mins resulted in the formation of suspension, which was filtered and dried to yield the final product (77% yield). MS: m/z 453 (M+1)⁺.

### Example 12

### 2-(2-(1-benzylpiperidin-4-ylidene)hydrazinyl)-4-(3-chlorophenyl)thiazole (3a)

The reaction of the 2-(1-benzylpiperidin-4-ylidene)hydrazinecarbothioamide (262 mg, 1 mM) and freshly synthesized 2-bromo-1-(3-chlorophenyl)ethanone (234 mg, 1 mM) in anhydrous THF at 30° C. for 60 mins resulted in the formation of suspension, which was filtered and dried to yield the final product (69% yield). ¹H NMR (300 MHz, CDCl₃): δ (ppm) 8.03 (s, 1H), 7.67 (d, 1H), 7.52 (d, 2H), 7.69 (d, 2H, J=1.98 Hz), 7.24-7.35 (m, 6H), 3.25 (s, 2H), 2.50 (m, 4H), 1.8 (m, 4H). IR (KBr) cm⁻¹: 3408, 3053, 2863, 2366, 1621, 1231, 773. MS: m/z 397 (M+1)⁺.

### Example 13

### 2-(2-(1-benzylpiperidin-4-ylidene)hydrazinyl)-4-(4-fluorophenyl)thiazole (3b)

The reaction of the 2-(1-benzylpiperidin-4-ylidene)hydrazinecarbothioamide (262 mg, 1 mM) and freshly synthesized 2-bromo-1-(4-fluorophenyl)ethanone (217 mg, 1 mM) in anhydrous THF at 35° C. for 60 mins in the formation of suspension, which was filtered and dried to yield the final product (73% yield). MS: m/z 381 (M+1)⁺.

### Example 14

### 2-(2-(1-benzylpiperidin-4-ylidene)hydrazinyl)-4-(3-nitrophenyl)thiazole (3c)

The reaction of the 2-(1-benzylpiperidin-4-ylidene)hydrazinecarbothioamide (262 mg, 1 mM) and freshly synthesized 2-bromo-1-(3-nitrophenyl)ethanone (244 mg, 1 mM) in anhydrous THF at 32° C. for 60 mins resulted in the formation of suspension, which was filtered and dried to yield the final product (78% yield). MS: m/z 408 (M+1)⁺.

### Example 15

### 2-(2-(1-benzylpiperidin-4-ylidene)hydrazinyl)-4-(4-methoxyphenyl)thiazole (3d)

The reaction of the 2-(1-benzylpiperidin-4-ylidene)hydrazinecarbothioamide (262 mg, 1 mM) and freshly synthesized 2-bromo-1-(4-methoxyphenyl)ethanone (229 mg, 1 mM) in anhydrous THF at 32° C. for 60 mins resulted in the formation of suspension, which was filtered and dried to yield the final product (83% yield). MS: m/z 393 (M+1)⁺.

### Example 16

### 2-(2-(1-benzylpiperidin-4-ylidene)hydrazinyl)-4-(pyridin-3-yl)thiazole (3e)

The reaction of the 2-(1-benzylpiperidin-4-ylidene)hydrazinecarbothioamide (262 mg, 1 mM) and freshly synthesized 2-bromo-1-(pyridin-3-yl)ethanone (200 mg, 1 mM) in anhydrous THF at 34° C. for 45 mins resulted in the formation of suspension, which was filtered and dried to yield the final product (82% yield). MS: m/z 364 (M+1)⁺.

### Example 17

### 2-(2-(1-benzylpiperidin-4-ylidene)hydrazinyl)-4-phenyl thiazole (3f)

The reaction of the 2-((1-benzyl-1H-indol-3-yl)methylene)hydrazinecarbothioamide (262 mg, 1 mM) and freshly synthesized 2-bromo-1-phenylethanone (199 mg, 1 mM) in anhydrous THF at 35° C. for 60 mins resulted in the formation of suspension, which was filtered and dried to yield the final product (79% yield). MS: m/z 363 (M+1)⁺.

### Example 18

### 2-(2-(1-(benzo[d][1,3]dioxol-5-yl)propan-2-ylidene)hydrazinyl)-4-(3-nitrophenyl)-thiazole (4a)

The reaction of the 2-(1-(benzo[d][1,3]dioxol-5-yl)propan-2-ylidene)hydrazinecarbothioamide (251 mg, 1 mM) and freshly synthesized 2-bromo-1-(3-nitrophenyl)ethanone (244 mg, 1 mM) in anhydrous THF at 32° C. for 90 mins resulted in the formation of suspension, which was filtered and dried to yield the final product (81% yield). ¹H NMR (300 MHz, CDCl₃): δ (ppm) 8.65 (s, 1H), 7.95 (m, 2H), 7.82 (m, 1H), 6.95 (s, 1H), 6.75 (dd, 2H), 5.58 (s, 2H), 2.45 (s, 2H), 2.1 (s, 3H). IR (KBr) cm⁻¹: 3408, 3053, 2863, 2366, 1621, 1231, 773. MS: m/z 397 (M+1)⁺.

### Example 19

### 2-(2-(1-(benzo[d][1,3]dioxol-5-yl)propan-2-ylidene)hydrazinyl)-4-(3-chlorophenyl)-thiazole (4b)

The reaction of the 2-(1-(benzo[d][1,3]dioxol-5-yl)propan-2-ylidene)hydrazinecarbothioamide (251 mg, 1 mM) and freshly synthesized 2-bromo-1-(3-chlorophenyl)ethanone (234 mg, 1 mM) in anhydrous THF at 32° C. for 60 mins resulted in the formation of suspension, which was filtered and dried to yield the final product (84% yield). MS: m/z 386 (M+1)⁺.

### Example 20

### 2-(2-(1-(benzo[d][1,3]dioxol-5-yl)propan-2-ylidene)hydrazinyl)-4-(4-methoxyphenyl)thiazole (4c)

The reaction of the 2-(1-(benzo[d][1,3]dioxol-5-yl)propan-2-ylidene)hydrazinecarbothioamide (251 mg, 1 mM) and freshly synthesized 2-bromo-1-(4-methoxyphenyl)ethanone (229 mg, 1 mM) in anhydrous THF at 32° C. for 60 mins resulted in the formation of suspension, which was filtered and dried to yield the final product (86% yield). MS: m/z 382 (M+1)⁺.

### Example 21

### 2-(2-(1-(benzo[d][1,3]dioxol-5-yl)propan-2-ylidene)hydrazinyl)-4-(4-fluorophenyl)-thiazole (4d)

The reaction of the 2-(1-(benzo[d][1,3]dioxol-5-yl)propan-2-ylidene)hydrazinecarbothioamide (251 mg, 1 mM) and freshly synthesized 2-bromo-1-(4-fluorophenyl)ethanone (217 mg, 1 mM) in anhydrous THF at 32° C. for 60 mins resulted in the formation of suspension, which was filtered and dried to yield the final product (75% yield). MS: m/z 370 (M+1)⁺.

### Example 22

### 2-(2-(1-(benzo[d][1,3]dioxol-5-yl)propan-2-ylidene)hydrazinyl)-4-phenyl thiazole (4e)

The reaction of the 2-(1-(benzo[d][1,3]dioxol-5-yl)propan-2-ylidene)hydrazinecarbothioamide (251 mg, 1 mM) and freshly synthesized 2-bromo-1-phenylethanone (199 mg, 1 mM) in anhydrous THF at 35° C. for 60 mins resulted in the formation of suspension, which was filtered and dried to yield the final product (78% yield). MS: m/z 352 (M+1)⁺.

### Example 23

### 1-(3-amino-4-((2-(4-(3-chlorophenyl)thiazol-2-yl)hydrazono)methyl)phenyl)ethanone (5a)

The reaction of the 2-(4-acetyl-2-aminobenzylidene)hydrazinecarbothioamide (236 mg, 1 mM) and freshly synthesized 2-bromo-1-(3-chlorophenyl)ethanone (234 mg, 1 mM) in anhydrous THF at 32° C. for 45 mins resulted in the formation of suspension, which was filtered and dried to yield the final product (81% yield). ¹H NMR (300 MHz, CDCl₃): δ (ppm) 8.23 (s, 1H), 7.92 (s, 1H), 7.88 (m, 2H), 7.49 (m, 3H), 7.35 (s, 1H), 6.36 (bs, NH₂), 2.58 (s, 3H). IR (KBr) cm⁻¹: 3408, 3060, 2863, 2356, 1725, 1621, 1231, 777. MS: m/z 371 (M+1)⁺.

### Example 24

### 1-(3-amino-4-((2-(4-(4-methoxyphenyl)thiazol-2-yl)hydrazono)methyl)phenyl)ethanone (5b)

The reaction of the 2-(4-acetyl-2-aminobenzylidene)hydrazinecarbothioamide (236 mg, 1 mM) and freshly synthesized 2-bromo-1-(4-methoxyphenyl)ethanone (230 mg, 1 mM) in anhydrous THF at 32° C. for 60 mins resulted in the formation of suspension, which was filtered and dried to yield the final product (76% yield). MS: m/z 367 (M+1)⁺.

### Example 25

### 1-(3-amino-4-((2-(4-(4-fluorophenyl)thiazol-2-yl)hydrazono)methyl)phenyl)ethanone (5c)

The reaction of the 2-(4-acetyl-2-aminobenzylidene)hydrazinecarbothioamide (236 mg, 1 mM) and freshly synthesized 2-bromo-1-(4-fluorophenyl)ethanone (217 mg, 1 mM) in anhydrous THF at 32° C. for 60 mins resulted in the formation of suspension, which was filtered and dried to yield the final product (84% yield). MS: m/z 355 (M+1)⁺.

### Example 26

### 1-(3-amino-4-((2-(4-(3-nitrophenyl)thiazol-2-yl)hydrazono)methyl)phenyl)ethanone (5d)

The reaction of the 2-(4-acetyl-2-aminobenzylidene)hydrazinecarbothioamide (236 mg, 1 mM) and freshly synthesized 2-bromo-1-(3-nitrophenyl)ethanone (244 mg, 1 mM) in anhydrous THF at 32° C. for 90 mins resulted in the formation of suspension, which was filtered and dried to yield the final product (79% yield). MS: m/z 382 (M+1)⁺.

### Example 27

### 1-(3-amino-4-((2-(4-phenyl thiazol-2-yl)hydrazono)methyl)phenyl)ethanone (5e)

The reaction of the 2-(4-acetyl-2-aminobenzylidene)hydrazinecarbothioamide (236 mg, 1 mM) and freshly synthesized 2-bromo-1-phenylethanone (199 mg, 1 mM) in anhydrous THF at 32° C. for 60 mins resulted in the formation of suspension, which was filtered and dried to yield the final product (83% yield). MS: m/z 337 (M+1)⁺.

### Example 28

### N,4-bis(4-fluorophenyl)thiazol-2-amine (6)

The reaction of 1-(4-fluorophenyl)thiourea (340, 2 mM) and freshly synthesized 2-bromo-1-(4-fluorophenyl)ethanone (435 mg, 2 mM) in anhydrous THF at room temperature for 45 mins resulted in the formation of suspension, which was filtered and dried to yield the final product (73% yield).

mp 127° C. ¹H NMR (300 MHz, CDCl₃): δ (ppm) 11.82 (s, 1H), 7.80 (m, 1H), 7.60 (m, 1H), 7.39 (m, 2H), 7.24 (m, 4H), 7.02 (m, 1H), 6.62 (s, 1H). IR (KBr) cm⁻¹: 3211, 3114, 2942, 2366, 1660, 1056. MS: m/z 289 (M+1)⁺. HRMS (ESI) m/z [M+1]⁺ calcd for C₁₅H₁₀F₂N₂S: 289.0533; found: 289.0491.

### Example 29

### N-(2,5-dimethoxyphenyl)-4-(4-fluorophenyl)thiazol-2-amine (7a)

The reaction of the 1-(4-fluorophenyl)thiourea (340, 2 mM) and freshly synthesized 2-bromo-1-(2,5-dimethoxyphenyl)ethanone (519 mg, 2 mM) in anhydrous THF at 32° C. for 60 mins resulted in the formation of suspension, which was filtered and dried to yield the final product (79% yield).

mp 160° C. ¹H NMR (300 MHz, CDCl₃): δ (ppm) 8.6 (s, 1H), 8.16 (d, 2H, J=9 Hz), 8.04 (d, 1H, J=2.7 Hz), 7.65 (s, 1H), 7.62 (t, 1H, J=7.98 Hz, 15.99 Hz), 6.86 (d, 1H, J=9 Hz), 6.58 (d, 1H, J=2.88 Hz), 6.56 (d, 1H, J=0.9 Hz). IR (KBr) cm⁻¹: 3507, 3076, 2938, 2366, 1578, 1022. MS: m/z 331 (M+1)⁺. HRMS (ESI) m/z [M+1]⁺ calcd for C₁₅H₁₀F₂N₂S: 331.0838; found: 289.0829.

### Example 30

### N-(2,5-dimethoxyphenyl)-4-(3-nitrophenyl)thiazol-2-amine (7b)

The reaction of the 1-(3-nitrophenyl)thiourea (395 mg, 2 mM) and freshly synthesized 2-bromo-1-(2,5-dimethoxyphenyl)ethanone (520 mg, 2 mM) in anhydrous THF at 30° C. for 45 mins resulted in the formation of suspension, which was filtered and dried to yield the final product (76% yield).

mp 154° C. MS: m/z 358 (M+1)⁺.

### Example 31

### N-(2,5-dimethoxyphenyl)-4-(4-methoxyphenyl)thiazol-2-amine (7c)

The reaction of the 1-(4-methoxyphenyl)thiourea (365 mg, 2 mM) and freshly synthesized 2-bromo-1-(2,5-dimethoxyphenyl)ethanone (520 mg, 2 mM) in anhydrous THF at 30° C. for 45 mins resulted in the formation of suspension, which was filtered and dried to yield the final product (74% yield).

mp 104° C. MS: m/z 343 (M+1)⁺.

### Example 32

### 4-(3,4-dimethoxyphenyl)-N-(2-(trifluoromethyl)phenyl)thiazol-2-amine (8a)

The reaction of the 1-(2-(trifluoromethyl)phenyl)thiourea (441 mg, 2 mM) and freshly synthesized 2-bromo-1-(3,4-dimethoxyphenyl)ethanone (520 mg, 2 mM) in anhydrous THF at 32° C. for 45 mins resulted in the formation of suspension, which was filtered and dried to yield the final product (73% yield).

mp 188° C. ¹H NMR (300 MHz, CDCl₃): δ (ppm) ¹H NMR (300 MHz, CDCl₃): δ (ppm) 7.85 (d, 1H, J=7.65), 7.73 (t, 2H, J=7.47 Hz, 11.19 Hz), 7.63 (d, 2H, J=7.23 Hz,), 7.38 (s, 1H), 7.32 (d, 2H, J=5.34), 6.96 (s, 1H), 3.99 (s, 6H). IR (Neat) cm⁻¹: 3463, 3080, 2968, 2366, 1589, 1023. MS: m/z 381 (M+1). HRMS (ESI) m/z calcd for C₁₈H₁₅Cl₂F₃N₂O₂S [M+1]⁺: 381.0806; found: 381.0820. HRMS (ESI) m/z [M+1]⁺ calcd for C₁₈H₁₅Cl₂F₃N₂O₂S: 381.0806; found: 381.0820.

### Example 33

### 4-(4-fluorophenyl)-N-(2-(trifluoromethyl)phenyl)thiazol-2-amine (8b)

The reaction of the 1-(2-(trifluoromethyl)phenyl)thiourea (441 mg, 2 mM) and freshly synthesized 2-bromo-1-(4-fluorophenyl)ethanone (434 mg, 2 mM) in anhydrous THF at 32° C. for 45 mins resulted in the formation of suspension, which was filtered and dried to yield the final product (78% yield). mp 184° C. MS: m/z 339 (M+1)⁺.

### Example 34

### 4-(3-nitrophenyl)-N-(2-(trifluoromethyl)phenyl)thiazol-2-amine (8c)

The reaction of the 1-(2-(trifluoromethyl)phenyl)thiourea (441 mg, 2 mM) and freshly synthesized 2-bromo-1-(3-nitrophenyl)ethanone (488 mg, 2 mM) in anhydrous THF at 35° C. for 45 mins resulted in the formation of suspension, which was filtered and dried to yield the final product (82% yield). mp 182° C. MS: m/z 366 (M+1)⁺.

### Example 35

### 4-(4-methoxyphenyl)-N-(2-(trifluoromethyl)phenyl)thiazol-2-amine (8d)

The reaction of the 1-(2-(trifluoromethyl)phenyl)thiourea (441 mg, 2 mM) and freshly synthesized 2-bromo-1-(4-methoxyphenyl)ethanone (458 mg, 2 mM) in anhydrous THF at 35° C. for 45 mins resulted in the formation of suspension, which was filtered and dried to yield the final product (79% yield). MS: m/z 351 (M+1)⁺.

### Example 36

### N-(4-fluorophenylcarbamothioyl)benzamide (B)

The equimolar amounts of 4-fluoroaniline and benzoyl isothiocyanate in dry benzene were stirred at 32° C. for 8 hrs. This led to formation of yellow precipitate which was left overnight at Room Temperature. Then, hexane (10 mL) was added and filtered to get light yellow solid which was dried and used in the next step. Yield 70%, mp 82° C.

### Example 37

### 1-(4-fluorophenyl)thiourea (C)

The N-(4-fluorophenylcarbamothioyl)benzamide (2.74 g) was refluxed in 10% NaOH aqueous solution (40 mL) at 100° C. for 1 hr. The reaction mixture was cooled and acidified with dilute HCl to get solid which was filtered and washed with water and dried. The dried solid was further recrystalized in dry ether to yield pure solid (92% yield). MS: m/z 1751 (M+1)⁺.

Advantages

The compound in the present invention represents a new class of potent anti-tuberculosis drugs with better anti-TB activity than the drug Nitazoxanide and thus may be potentially effective for the treatment of MDR-TB and XDR-TB. In addition, the compounds in the present invention may be beneficial for TB programs that need to ensure optimal patient adherence throughout the entire treatment course.

1. The compound of general formula A, 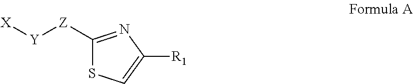
Wherein;
R₁ is substituted/unsubstituted aryl or heteroaryl group of the structure 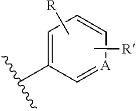
wherein,
A is CH or N
R and R′ are groups, which may be identical or different, selected from the group consisting of hydrogen, halogen, nitro, and methoxy.
X is a group selected from the group consisting of 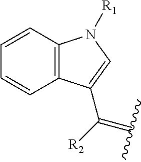
wherein, R₁ is a group selected from the group of benzyl and 2,4-dichlorobenzyl, while R2 is a group selected from hydrogen and methyl.
or, a group of structure 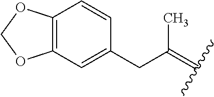 [SMILES: *=C(C)Cc1ccc2c(c1)OCO2]
or, a group of structure 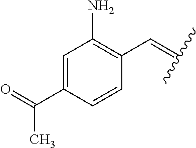 [SMILES: *=Cc1ccc(C(C)=O)cc1N]
or, a group of structure 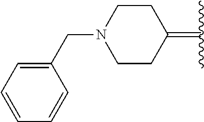 [SMILES: *=C1CCN(Cc2ccccc2)CC1]
or a group of structure 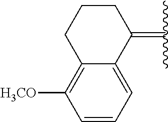 [SMILES: *=C1CCCc2c(OC)cccc21]
or a group of structure 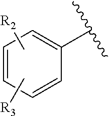
wherein, R2 and R3 may be same or different selected from the group of hydrogen, halogen, methoxy, trifluoromethyl.
Y is tertiary N like ═N or may be absent in cases where the X is directly attached to Z
the bond between X and Z is single in cases where Y is absent and X is directly attached to Z
the bond between X and Y is double, when Y is present
Z=is NH.

2. The compound of general formula A as claimed in claim 1 wherein, the chemical formula of the representative compounds comprising:
4-(3,4-dimethoxyphenyl)-2-(2-(5-methoxy-3,4-dihydronaphthalen-1(2H)-ylidene)hydrazinyl)thiazole (1a)
4-(2,4-dichlorophenyl)-2-(2-(5-methoxy-3,4-dihydronaphthalen-1(2H)-ylidene)hydrazinyl)thiazole (1b)
4-(4-fluorophenyl)-2-(2-(5-methoxy-3,4-dihydronaphthalen-1(2H)-ylidene)hydrazinyl)thiazole (1c)
2-(2-(5-methoxy-3,4-dihydronaphthalen-1(2H)-ylidene)hydrazinyl)-4-(3-nitrophenyl)thiazole (1d)
2-(2-((1-(2,4-dichlorobenzyl)-1H-indol-3-yl)methylene)hydrazinyl)-4-(3,4-dimethoxyphenyl)thiazole (2a)
2-(2-((1-(2,4-dichlorobenzyl)-1H-indol-3-yl)methylene)hydrazinyl)-4-(2,4-dichlorophenyl)thiazole (2b)
2-(2-((1-(2,4-dichlorobenzyl)-1H-indol-3-yl)methylene)hydrazinyl)-4-(4-fluorophenyl)-thiazole (2c)
2-(2-(1-(1-benzyl-1H-indol-3-yl)ethylidene)hydrazinyl)-4-(3-chlorophenyl)-thiazole. (2d)
2-(2-(1-(1-benzyl-1H-indol-3-yl)ethylidene)hydrazinyl)-4-(4-fluorophenyl)-thiazole. (2e)
2-(2-(1-(1-benzyl-1H-indol-3-yl)ethylidene)hydrazinyl)-4-phenylthiazole. (2f) 2-(2-(1-(1-benzyl-1H-indol-3-yl)ethylidene)hydrazinyl)-4-(4-methoxyphenyl)-thiazole. (2g)
2-(2-(1-benzylpiperidin-4-ylidene)hydrazinyl)-4-(3-chlorophenyl)-thiazole. (3a)
2-(2-(1-benzylpiperidin-4-ylidene)hydrazinyl)-4-(4-fluorophenyl)-thiazole. (3b)
2-(2-(1-benzylpiperidin-4-ylidene)hydrazinyl)-4-(3-nitrophenyl)-thiazole. (3c)
2-(2-(1-benzylpiperidin-4-ylidene)hydrazinyl)-4-(4-methoxyphenyl)-thiazole. (3d)
2-(2-(1-benzylpiperidin-4-ylidene)hydrazinyl)-4-(pyridin-3-yl)-thiazole. (3e)
2-(2-(1-benzylpiperidin-4-ylidene)hydrazinyl)-4-phenylthiazole. (3f)
2-(2-(1-(benzo[d][1,3]dioxol-5-yl)propan-2-ylidene)hydrazinyl)-4-(3-nitrophenyl)-thiazole. (4a)
2-(2-(1-(benzo[d][1,3]dioxol-5-yl)propan-2-ylidene)hydrazinyl)-4-(3-chlorophenyl)-thiazole. (4b)
2-(2-(1-(benzo[d][1,3]dioxol-5-yl)propan-2-ylidene)hydrazinyl)-4-(4-methoxyphenyl)-thiazole. (4c)
2-(2-(1-(benzo[d][1,3]dioxol-5-yl)propan-2-ylidene)hydrazinyl)-4-(4-fluorophenyl)-thiazole. (4d)
2-(2-(1-(benzo[d][1,3]dioxol-5-yl)propan-2-ylidene)hydrazinyl)-4-phenylthiazole. (4e)
1-(3-amino-4-((2-(4-(3-chlorophenyl)thiazol-2-yl)hydrazono)methyl)phenyl)ethanone. (5a)
1-(3-amino-4-((2-(4-(4-methoxyphenyl)thiazol-2-yl)hydrazono)methyl)phenyl)ethanone. (5b)
1-(3-amino-4-((2-(4-(4-fluorophenyl)thiazol-2-yl)hydrazono)methyl)phenyl)ethanone. (5c)
1-(3-amino-4-((2-(4-(3-nitrophenyl)thiazol-2-yl)hydrazono)methyl)phenyl)ethanone. (5d)
1-(3-amino-4-((2-(4-phenyl thiazol-2-yl)hydrazono)methyl)phenyl)ethanone. (5e)
N,4-bis(4-fluorophenyl)thiazol-2-amine (6)
N-(2,5-dimethoxyphenyl)-4-(4-fluorophenyl)thiazol-2-amine (7a)
N-(2,5-dimethoxyphenyl)-4-(3-nitrophenyl)thiazol-2-amine (7b)
N-(2,5-dimethoxyphenyl)-4-(4-methoxyphenyl)thiazol-2-amine (7c)
4-(3,4-dimethoxyphenyl)-N-(2-(trifluoromethyl)phenyl)thiazol-2-amine (8a)
4-(4-fluorophenyl)-N-(2-(trifluoromethyl)phenyl)thiazol-2-amine (8b)
4-(3-nitrophenyl)-N-(2-(trifluoromethyl)phenyl)thiazol-2-amine (8c)
4-(4-methoxyphenyl)-N-(2-(trifluoromethyl)phenyl)thiazol-2-amine (8d)

3. The compound of general formula A as claimed in claim 1, wherein the structural formula of representative compounds comprising:

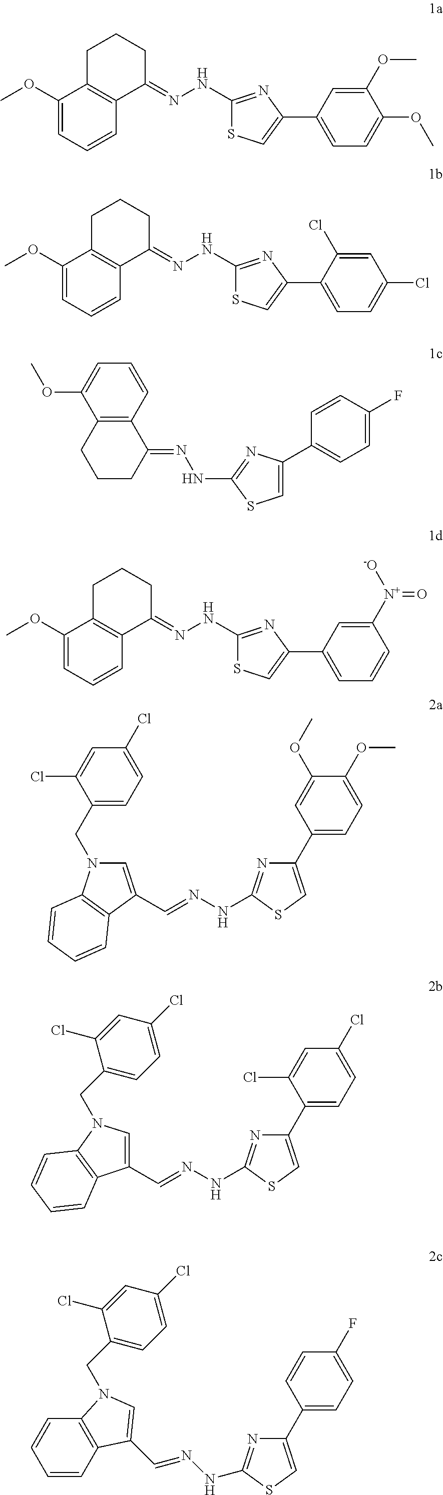

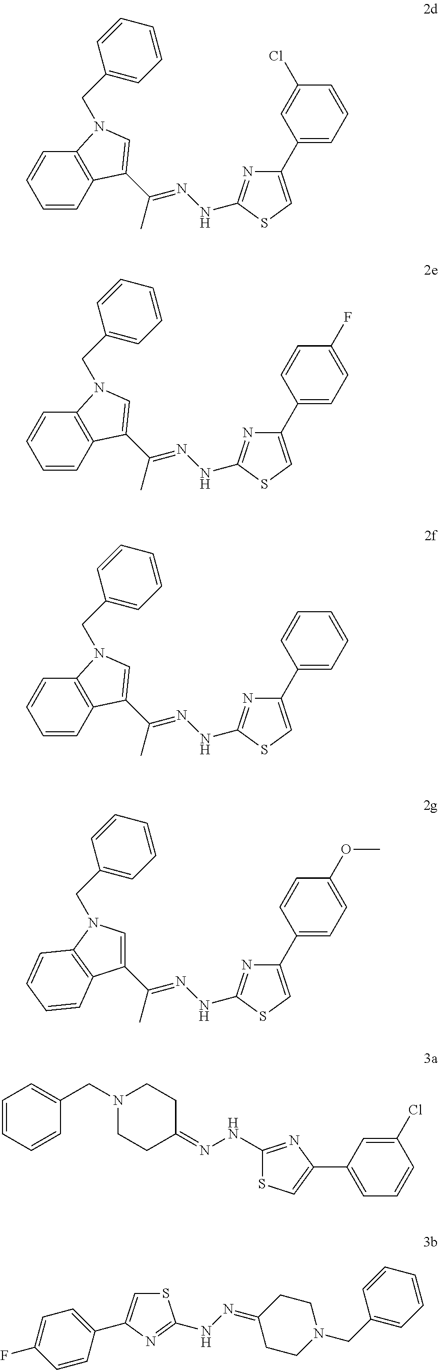

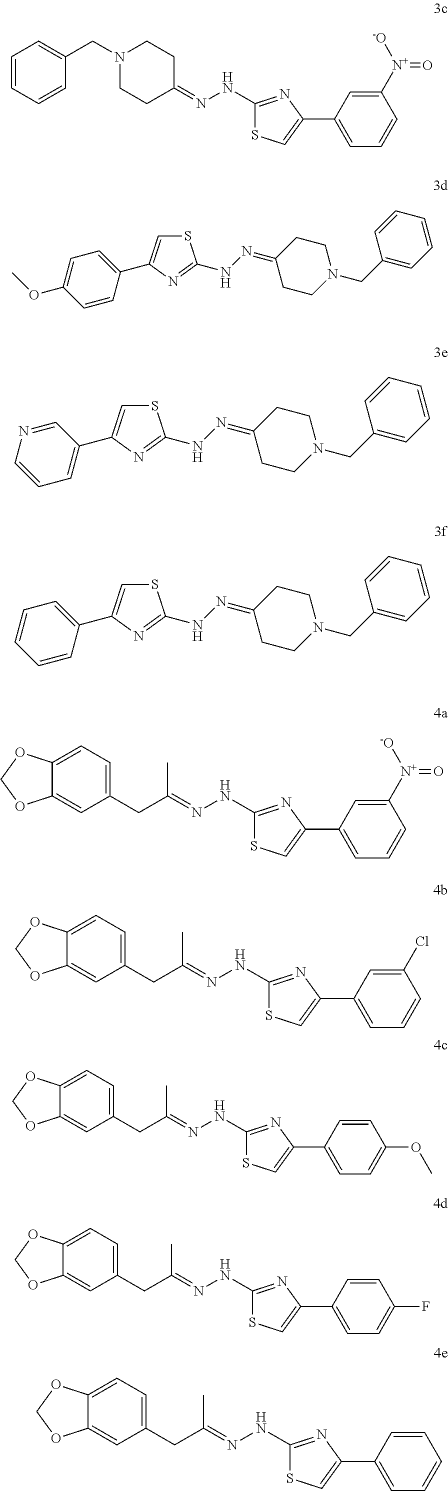

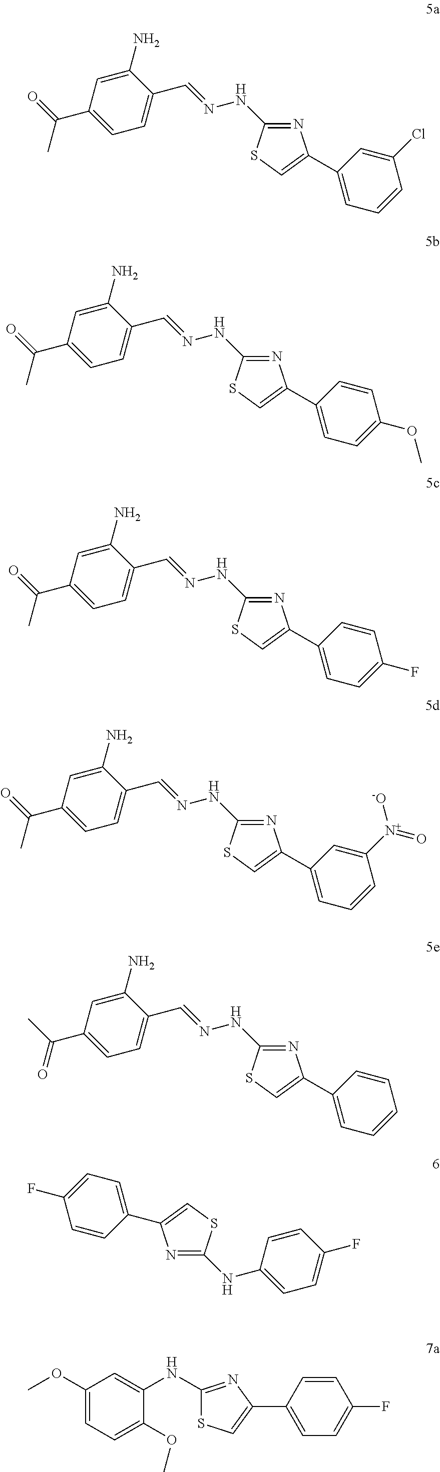

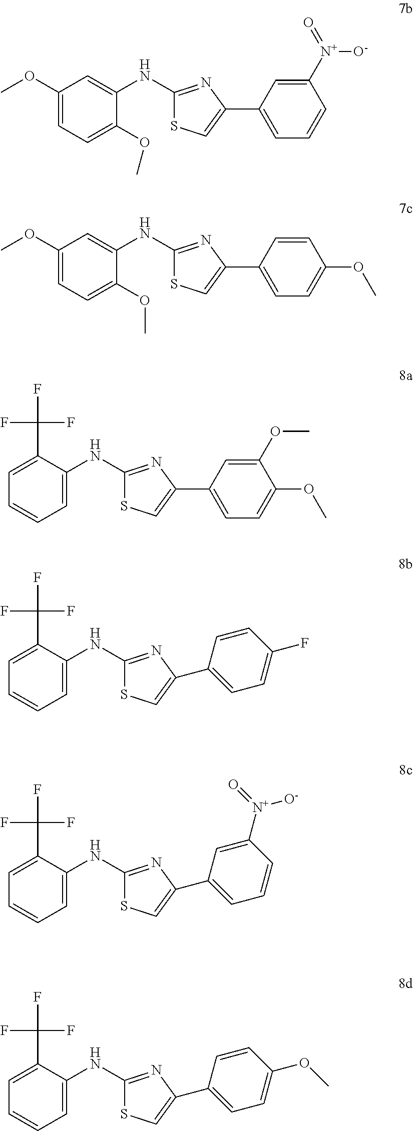

4. The Compound of general formula A as claimed in claim 1, wherein said compounds are useful as anti-tuberculosis agent particularly in the treatment of multi-drug resistant tuberculosis (MDR-TB) and extensively drug resistant tuberculosis (XDR-TB).

5. The compound of the general formula A as claimed in claim 1, wherein said compounds exhibit MIC in the range of 6.25 to 31.73 μM causing 90% growth inhibition.

6. A process for the synthesis of compounds of general formula A as claimed in claim 1, wherein the said process comprising:
reacting substituted/unsubstituted alpha-bromoacetophenone with substituted phenylthiourea represented by formula C wherein R2 is selected from a group consisting of fluoro, methoxy, nitro, trifluoromethyl, or substituted hydrazine carbothioamide represented by formula D wherein X is selected from groups as described in claim 1, in a solvent selected from a group 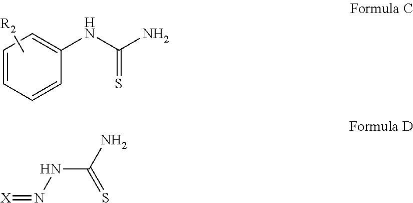
consisting of anhydrous THF, acetone, ethanol, or other nonpolar/polar solvents at a temperature ranging between 10° C. to 60° C. for a period ranging between 0.5 hr to 24 hrs to provide compounds of general formula A.

7. A process as claimed in claim 6 wherein, the alpha-bromo acetophenone is selected from a group consisting of 2-bromo-1-(3,4-dimethoxyphenyl)ethanone, 2-bromo-1-(2,4-dichlorophenyl)ethanone, 2-bromo-1-(4-fluorophenyl)ethanone, 2-bromo-1-(3-nitrophenyl)ethanone, 2-bromo-1-(3-chlorophenyl)ethanone, 2-bromo-1-phenylethanone, 2-bromo-1-(4-methoxyphenyl)ethanone, 2-bromo-1-(3-chlorophenyl)ethanone, 2-bromo-1-(4-fluorophenyl)ethanone, 2-bromo-1-(3-nitrophenyl)ethanone, 2-bromo-1-(4-methoxyphenyl)ethanone, 2-bromo-1-(pyridin-3-yl)ethanone, 2-bromo-1-phenylethanone, 2-bromo-1-(3-nitrophenyl)ethanone, 2-bromo-1-(3-chlorophenyl)ethanone, 2-bromo-1-(4-methoxyphenyl)ethanone, 2-bromo-1-(4-fluorophenyl)ethanone, 2-bromo-1-phenylethanone, 2-bromo-1-(3-chlorophenyl)ethanone, 2-bromo-1-(4-fluorophenyl)ethanone, 2-bromo-1-(3-nitrophenyl)ethanone, 2-bromo-1-(3,4-dimethoxyphenyl)ethanone, 2-bromo-1-(2,5-dimethoxyphenyl)ethanone, or 2-bromo-1-(4-fluorophenyl)ethanone

8. A process as claimed in claim 6 wherein the substituted hydrazine carbothioamide is selected from the group consisting of 2-(5-methoxy-3,4-dihydronaphthalen-1 (2H)-ylidene)hydrazinecarbothioamide, 2-((1-(2,4-dichlorobenzyl)-1H-indol-3-yl)methylene)hydrazine carbothioamide, 2-((1-benzyl-1H-indol-3-yl)methylene)hydrazine-carbothioamide, 2-(1-benzylpiperidin-4-ylidene)hydrazinecarbothioamide, -(1-(benzo[d][1,3]dioxol-5-yl)propan-2-ylidene)hydrazinecarbothioamide, or 2-(4-acetyl-2-aminobenzylidene)hydrazine carbothioamide.

9. A process as claimed in claim 6 wherein the substituted phenyl thiourea is selected from a group consisting of 1-(4-fluorophenyl)thiourea, 1-(3-nitrophenyl)thiourea, 1-(4-methoxyphenyl)thiourea, 1-(2-(trifluoromethyl)phenyl)thiourea, 1-(2-(trifluoromethyl)phenyl)thiourea, 1-(2-(trifluoromethyl)phenyl)thiourea, or 1-(2-(trifluoromethyl)phenyl)thiourea.

10. The process as claimed in claim 6, wherein a method of preparation of substituted phenyl thiourea comprises:
(a) reacting substituted aniline with benzoyl isothiocyanate in dry benzene for 8-10 hrs to afford the corresponding N-(substitutedphenylcarbamothioyl)-benzamide represented by formula B, wherein R2 and R3 may be same or different selected from a group consisting of hydrogen, fluoro, methoxy, nitro, trifluoromethyl, 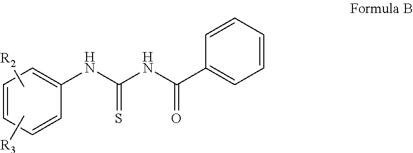
(b) debenzoylating compounds of formula B as obtained in step (a) by refluxing in 10% NaOH aqueous solution at 100° C. for 1 hr to afford the corresponding phenylthiourea of formula C wherein R2 and R3 may be same or different selected from a group consisting of hydrogen, fluoro, methoxy, nitro, trifluoromethyl.

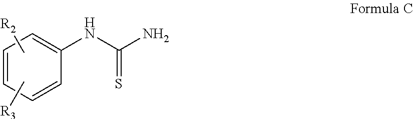

11. The process as claimed in claim 9, wherein method of preparation of substituted phenyl thiourea comprises:
(a) reacting substituted aniline with benzoyl isothiocyanate in dry benzene for 8-10 hrs to afford the corresponding N-(substitutedphenylcarbamothioyl)-benzamide represented by formula B, wherein R2 and R3 may be same or different selected from a group consisting of hydrogen, fluoro, methoxy, nitro, trifluoromethyl, 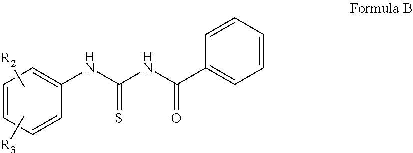
(b) debenzoylating compounds of formula B as obtained in step (a) by refluxing in 10% NaOH aqueous solution at 100° C. for 1 hr to afford the corresponding phenylthiourea of formula C wherein R2 and R3 may be same or different selected from a group consisting of hydrogen, fluoro, methoxy, nitro, trifluoromethyl. [chemical structure]
